(19) United States
(12) Patent Application Publication
(10) Pub. No.: US 2022/0062487 A1
(43) Pub. Date: Mar. 3, 2022

(54) SYSTEMS AND METHODS FOR CONTROLLING INDOOR AIR QUALITY WITH A FLUID MOVING APPARATUS

(71) Applicant: Regal Beloit America, Inc., Beloit, WI (US)

(72) Inventors: Roger Carlos Becerra, Fort Wayne, IN (US); William Stuart Gatley, Cassville, MO (US); William Arthur Ziegler, Jefferson City, MO (US); Mohamad Khalil Dahouk, Fort Wayne, IN (US); Michael David Smith, Rogers, AR (US); Audwin William Cash, Fort Wayne, IN (US); Zachary Joseph Stauffer, Fort Wayne, IN (US); Brian Lee Beifus, Fort Wayne, IN (US); John Carl Kunze, Fort Wayne, IN (US); Ramin Rezaei, Centerville, OH (US); Joseph A. Henry, Dayton, OH (US)

(21) Appl. No.: 17/320,908

(22) Filed: May 14, 2021

Related U.S. Application Data
(63) Continuation-in-part of application No. 17/100,326, filed on Nov. 20, 2020.
(60) Provisional application No. 62/706,561, filed on Aug. 25, 2020.

(51) Int. Cl. A61L 9/20; A61L 9/22

(52) U.S. Cl. CPC A61L 9/20; A61L 9/22; A61L 2209/111; A61L 2209/14; A61L 2209/12; A61L 2209/16

(57) ABSTRACT

One aspect of the disclosure includes a fluid moving system. The fluid moving system includes a fluid moving apparatus configured to convey a fluid through a housing from an inlet to an outlet. The fluid moving system includes an active cleaning device configured to neutralize or remove at least a portion of an undesired matter from the fluid conveyed through the housing. The fluid moving system includes an electric motor including a rotor coupled to the fluid moving apparatus and configured to turn the fluid moving apparatus upon application of electric power to a stator of the electric motor. The fluid moving system includes a motor controller communicatively coupled to the electric motor and configured to control at least one of a speed output or a torque output thereof.

### CROSS-REFERENCE TO RELATED APPLICATIONS

The present application is a continuation-in-part of U.S. patent application Ser. No. 17/100,326, filed Nov. 20, 2020, and entitled “SYSTEMS AND METHODS FOR CONTROLLING INDOOR AIR QUALITY WITH A FLUID MOVING APPARATUS,” which claims the benefit of and priority to U.S. Provisional Patent Application Ser. No. 62/706,561, filed Aug. 25, 2020, and entitled “SYSTEMS AND METHODS FOR CONTROLLING INDOOR AIR QUALITY WITH A FLUID MOVING APPARATUS,” the disclosures of which are hereby incorporated by reference in their entirety.

### FIELD

The field of the disclosure relates generally to a fluid moving system and, more specifically, a fluid moving apparatus for controlling indoor air quality (IAQ).

### BACKGROUND

Indoor air quality (IAQ) generally refers to the air quality within and around buildings and structures, such as, for example, single-family homes, apartments, commercial and industrial buildings, or office buildings. IAQ is typically determined by collecting and testing air samples, monitoring human exposure to pollutants, or collection and testing of samples or deposits on building surfaces. Computer modeling of air flow within and around buildings may also be incorporated into an IAQ determination.

IAQ can be quantified in various measurable ways. For example, by identifying the existence of particulate matter, volatile organic compounds (VOCs), carbon monoxide, carbon dioxide, or other airborne pollutants. Temperature and humidity conditions can also correlate to IAQ. Moreover, the existence of specific biologic matter, such as airborne bacterial or viral particulates, can impact IAQ and the functional utility of a given space.

IAQ can conventionally be maintained in and around a given indoor space with the addition of disinfectant, sterilizing, filtering, purifying, or other air processing systems to mitigate airborne pollutants.

### BRIEF DESCRIPTION

One aspect of the disclosure includes a fluid moving system. The fluid moving system includes a fluid moving apparatus configured to convey a fluid through a housing from an inlet to an outlet. The fluid moving system includes an active cleaning device configured to neutralize or remove at least a portion of an undesired matter from the fluid conveyed through the housing. The fluid moving system includes an electric motor including a rotor coupled to the fluid moving apparatus and configured to turn the fluid moving apparatus upon application of electric power to a stator of the electric motor. The fluid moving system includes a motor controller communicatively coupled to the electric motor and configured to control at least one of a speed output or a torque output thereof.

Another aspect of the disclosure includes a method for operating a fluid moving system. The method includes conveying, using a fluid moving apparatus, a fluid through a housing from an inlet to an outlet. The method further includes removing, using an active cleaning device, at least a portion of an undesired matter from the fluid conveyed through the housing. The method further includes turning, using an electric motor including a rotor coupled to the fluid moving apparatus, the fluid moving apparatus upon application of electric power to a stator of the electric motor. The method further includes controlling, using a motor controller communicatively coupled to the electric motor, at least one of a speed output or a torque output of the electric motor.

Yet another aspect of the disclosure includes a heating, ventilation, and air conditioning (HVAC) system. The HVAC system includes a fluid conduit and a fluid moving system coupled in flow communication with said fluid conduit. The fluid moving system includes a fluid moving apparatus configured to convey a fluid through a housing from an inlet to an outlet. At least one of the inlet and the outlet is coupled in flow communication with said fluid conduit. The fluid moving system further includes an active cleaning device configured to neutralize or remove at least a portion of an undesired matter from the fluid conveyed through the housing. The fluid moving system further includes an electric motor including a rotor coupled to the fluid moving apparatus and configured to turn the fluid moving apparatus upon application of electric power to a stator of the electric motor. The fluid moving system further includes a motor controller communicatively coupled to said electric motor and configured to control at least one of a speed output or a torque output thereof.

### BRIEF DESCRIPTION OF THE DRAWINGS

FIG. 1 is a perspective diagram of an example fluid moving system including an active cleaning device;

FIG. 2 is a perspective diagram of another example fluid moving system including an active cleaning device;

FIG. 3 is another perspective diagram of the fluid moving system shown in FIG. 1;

FIG. 4 is another perspective diagram of the fluid moving system shown in FIGS. 1 and 3;

FIG. 5 is a perspective diagram of an example active cleaning device for use with the fluid moving apparatuses of FIGS. 1-4;

FIG. 6 is another perspective diagram of the active cleaning device shown in FIG. 5;

FIG. 7 is a schematic diagram of an example blower housing having a baffle on which an active cleaning device is mounted;

FIG. 8 is a perspective diagram of the blower housing shown in FIG. 7;

FIG. 9 is a perspective diagram of another example blower housing having a baffle on which an active cleaning device may be mounted;

FIG. 10 is a disassembled view of an example electronics module for an electric motor including an indoor air quality sensor;

FIG. 11 is a schematic diagram of the electronics module shown in FIG. 10;

FIG. 12 is a schematic diagram of an example electric motor having an indoor air quality sensor;

FIG. 13 is a schematic diagram of an example indoor air quality sensor;

FIG. 14 is a graphical illustration of an example user interface for a system controller for use with the fluid moving systems, active cleaning devices, and electric motors shown in FIGS. 1-12;

FIG. 15 is a graphical illustration of another example user interface for a system controller for use with the fluid moving systems, active cleaning devices, and electric motors shown in FIGS. 1-12;

FIG. 16 is a flowchart of an exemplary method for operating a blower system;

FIG. 17 is a perspective diagram of another example fluid moving system;

FIG. 18 is another perspective diagram of the example fluid moving system shown in FIG. 17;

FIG. 19 is a perspective diagram of another example fluid moving system;

FIG. 20 is another perspective diagram of the example fluid moving system shown in FIG. 19;

FIG. 21 is an example ionizer and ultraviolet (UV) combination unit for use with the fluid moving systems shown in FIGS. 19 and 20;

FIG. 22 is a perspective view of an example UV lamp;

FIG. 23 is a perspective view of an example fluid treatment system;

FIG. 24 is another perspective diagram of the example fluid treatment system shown in FIG. 23;

FIG. 25, is another perspective diagram of the example fluid treatment system shown in FIGS. 23 and 24;

FIG. 26 is another perspective diagram of the example fluid treatment system shown in FIGS. 23-25;

FIG. 27 is another perspective diagram of the example fluid treatment system shown in FIGS. 22-26;

FIG. 28 is a perspective diagram of an example air scrubber unit;

FIG. 29 is a perspective diagram of an example UV emitter for use with the example fluid treatment system shown in FIGS. 22-27;

FIG. 30 is another perspective diagram of the example UV emitter shown in FIG. 29;

FIG. 31 is a flowchart of an exemplary method for operating a fluid treatment system;

FIG. 32 is a perspective diagram of another example fluid moving system;

FIG. 33 is another perspective diagram of the example fluid moving system shown in FIG. 32; and

FIG. 34 is another perspective diagram of the example fluid moving system shown in FIGS. 32 and 33.

### DETAILED DESCRIPTION

Embodiments of the disclosed fluid moving system include a fluid moving apparatus integrated with an IAQ sensor and an active cleaning device, such as an Ultra Violet (UV) light source (e.g., UV-A, UV-B, or UV-C), ion generator, or electrostatic filtration device. An embodiment system includes, for example, a blower configured to move a fluid, such as air, from an inlet though an outlet of a fan housing, and an electric motor configured to turn the blower. The electric motor is a variable speed motor that enables, for example, continuously variable speed or discrete speed settings. Further, the electric motor may include an induction motor, a permanent split capacitor (PSC) motor, an electrically commutated motor (ECM), or any other suitable electric motor for operating the blower. The blower may include forward-curved, backward-curved, or radial blades. In alternative embodiments, the fluid moving apparatus includes a fan. In further alternative embodiments, the disclosed fluid moving system may utilize the active cleaning device to improve the quality of another fluid, such as water, and where the fluid moving apparatus includes an impeller. The disclosed system further includes a motor controller configured to control operation of the motor based on commands received from a system controller (e.g., a thermostat, a heating, ventilation, and air conditioning (HVAC) unit controller, or other computing system) and based on data received from sensors in communication with the motor controller. The fluid moving system, in certain embodiments, further includes an ultraviolet light source (sometimes referred to herein as a “UV unit”) configured to emit UV radiation though air moving through the air moving system. The UV unit is configured for communication with and may be controlled by the motor controller. The integration of the active cleaning device onto the fluid moving system enables the cleansing of all circulated fluid at a central location. Moreover, circulated air can be monitored (e.g., by an IAQ sensor) and used as a feedback to control the speed and quantity of fluid moving across the active cleaning device to better regulate the cleaning effect of the active cleaning device.

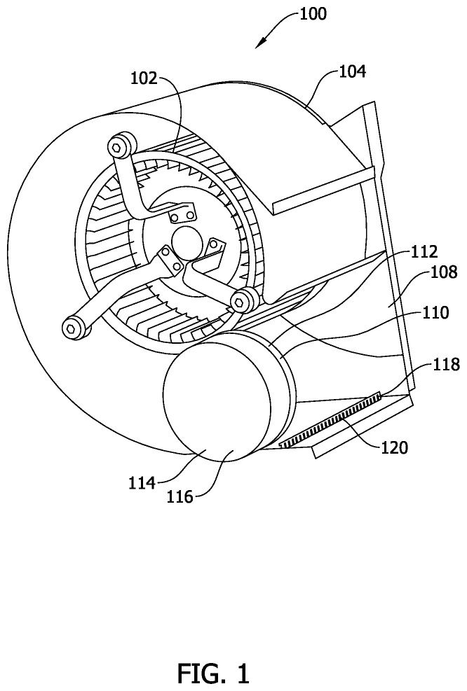

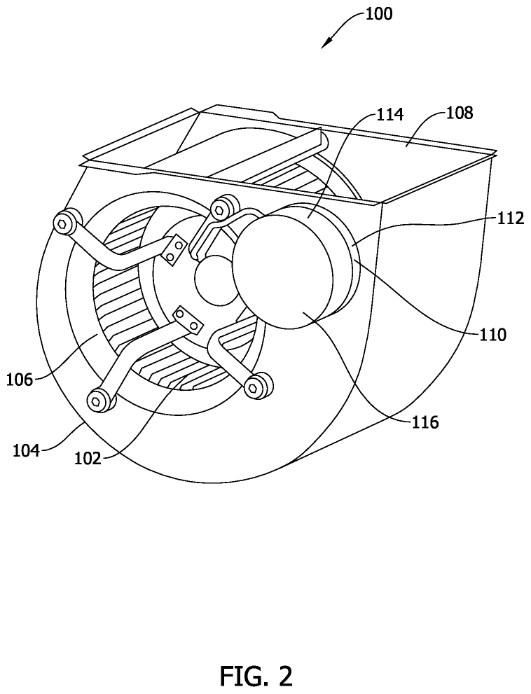

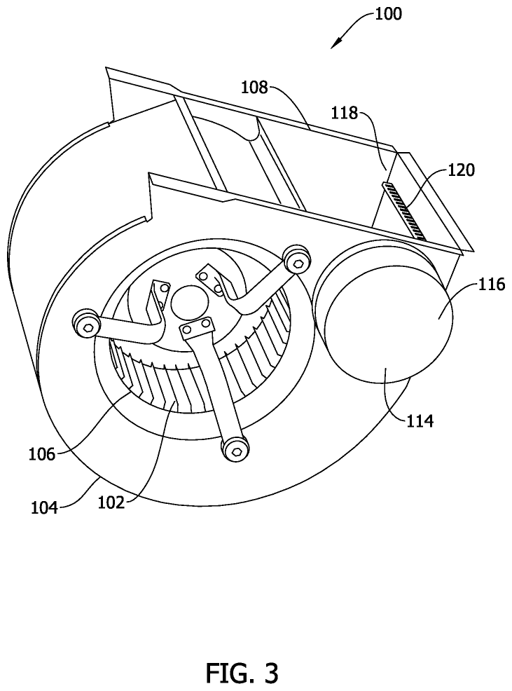

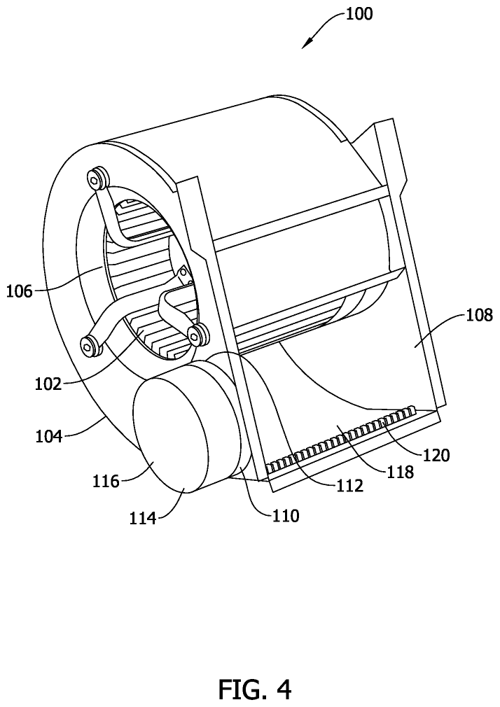

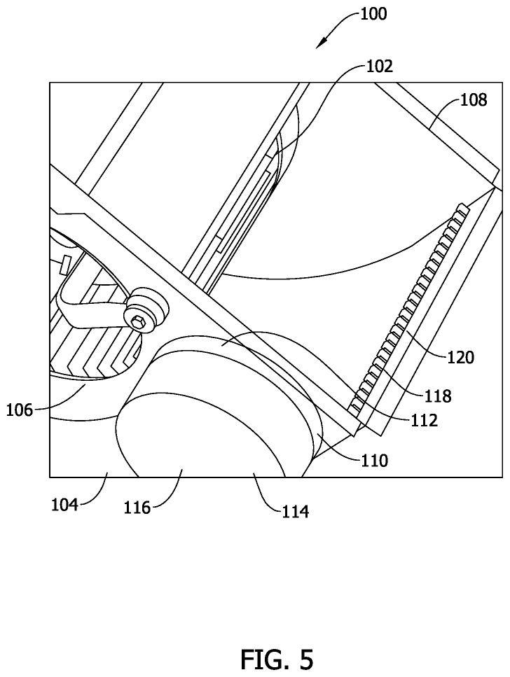

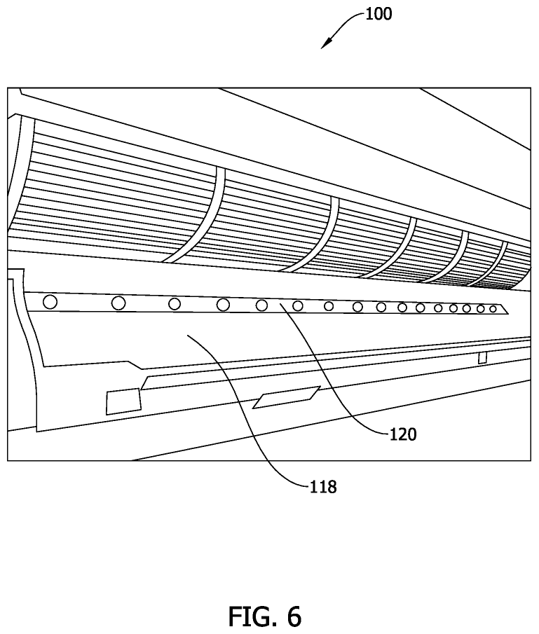

FIG. 1 is a partial cross-sectional view of an exemplary fluid moving apparatus and, more specifically, a blower system 100 configured to control indoor air quality (IAQ). Blower system 100 includes a blower wheel 102 disposed in a blower housing 104 having an inlet 106 and an outlet 108. Blower system 100 further includes an electric motor 110 configured to turn blower wheel 102 to cause a fluid such as air to move into blower housing 104 through inlet 106 and out of the blower housing 104 through outlet 108. In some embodiments, inlet 106 and outlet 108 are coupled in flow communication with an HVAC system.

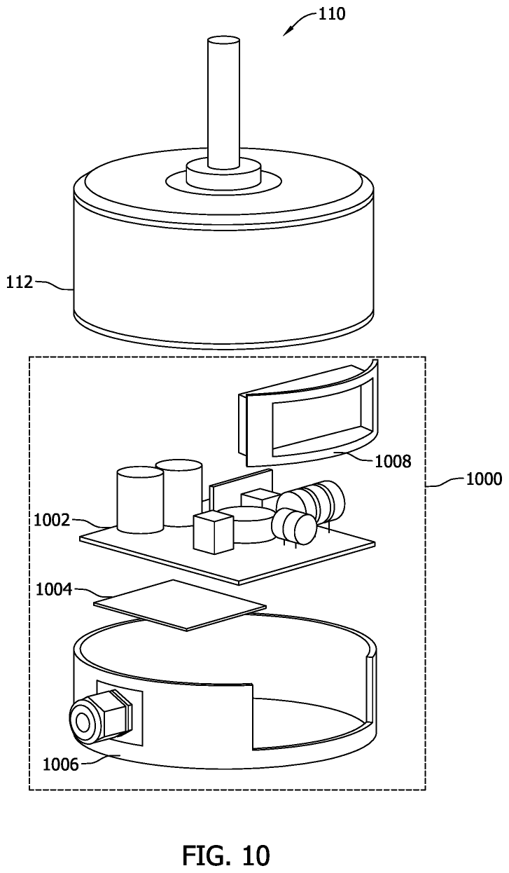

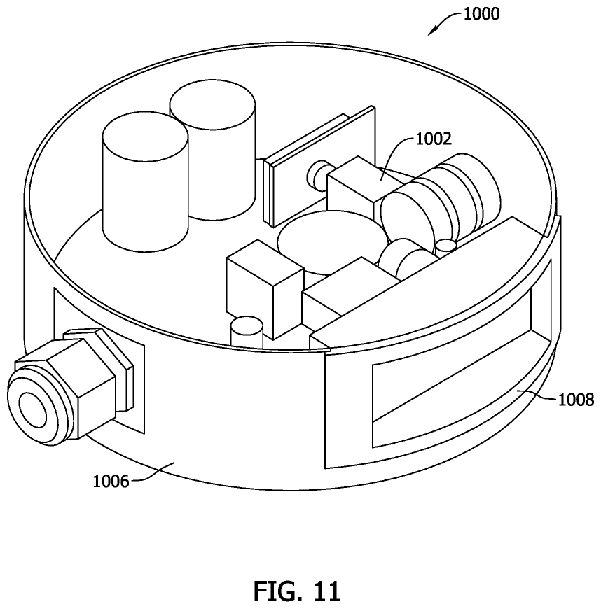

Electric motor 110 includes a rotor and a stator (not shown). In some embodiments, the rotor and the stator are disposed in a motor housing 112. The rotor is coupled to blower wheel 102 via a shaft (not shown), and is configured to rotate in response to a current present in windings of the stator. Electric motor 110 further includes a motor controller 114 configured to supply current to the windings of the stator to cause blower wheel 102 to rotate. Motor controller 114 is typically incorporated with the electric motor 110 and within motor housing 112 in an electronics enclosure (as shown in FIG. 10) or module. Alternatively, motor controller 114 may be external to electric motor 110 and positioned within a unit of equipment in which electric motor 110 is installed. Motor controller 114 may also be remote from electric motor 110 or the unit of equipment in which the motor is installed. For example, at least some motor control functions may be implemented on a remote control device or remote computing device external to electric motor 110 and external to the unit of equipment. In certain embodiments, certain components of motor controller 114 may be local to electric motor 110 or within motor housing 112, and other components may be external or remote. Likewise, some motor control functions may be embodied on local motor controller components (e.g., local power electronics or local digital electronics), and other motor control functions may be embodied on remote or external motor controller components (e.g., remote power electronics or remote digital electronics).

Motor controller 114 includes a processor (shown in FIG. 11) and an inverter configured to control the supply of current to the stator windings based on instruction from the processor. In some embodiments, motor controller 114 is configured for communication with a system controller (not shown), such as a thermostat, HVAC system controller, or other suitable computing device, such as, for example, a personal computer, laptop, smart phone, tablet computer, server, or cloud computing platform. In such embodiments, motor controller 114 may be configured, e.g., programmed or loaded with executable instructions, to operate blower system 100 based on, for example, a speed, torque, or airflow command received from the system controller. Electric motor 110 then operates, for example, by speed control, torque control, constant airflow, or constant mass flow. In some embodiments, motor controller 114 is disposed in a motor controller housing 116 that may be disposed adjacent to, attached to, or integrated with the motor housing 112. In some embodiments, blower system 100 is configured for self-cleaning or operation that minimizes the accumulation of dust on blower wheel 102 or within the blower system 100. For example, blower wheel 102 may include backward-curved blades, which resist the accumulation of dust.

The blower system 100 further includes at least one UV unit 118 configured to emit UV light capable of influencing an air quality of air moving through the fluid moving system. For example, the UV light may improve the air quality by killing pathogens or removing unwanted particles present in the air moving through the blower system 100. Accordingly, when coupled to an HVAC system, the blower system 100 can control an IAQ of a space treated by the HVAC system.

UV unit 118 is configured for communication with the processor of the motor controller 114, such that UV unit 118 responds to a control signal generated by motor controller 114. In response to the control signal, UV unit 118 is configured, for example, to activate, deactivate, or change an intensity of the UV light. For example, in some embodiments, UV motor controller 114 may activate UV unit 118 when a detected level of contaminants is greater than a threshold, or vary the intensity of the UV light as a function of the detected level. In some embodiments, UV unit 118 includes one or more light emitting diodes (LEDs) 120 or other elements configured to emit UV radiation in response to an electric signal. In some embodiments, the UV unit 118 is configured to receive power from a power supply of the motor controller, for example, via a wired direct current (DC) bus. Additionally or alternatively, UV unit 118 may be powered by an internal source, such as a battery.

In some embodiments, blower system 100 includes a turbine device capable of generating electrical power from the fluid flow through blower system 100. In such embodiments, the turbine device may be electrically coupled to and provide power to UV unit 118 or other electrical components of blower system 100. Additionally or alternatively, in certain embodiments, blower system 100 includes, or is coupled to, a photovoltaic cell or other device capable of generating electrical power from a light source, such as the sun, artificial lighting, or other ambient light. In such embodiments, the photovoltaic cell is positioned to receive light from the light source and is electrically coupled to and provides power to UV unit 118 or other electrical components of blower system 100. In certain embodiments, blower system 100 includes, or is coupled to, a thermopile or other device capable of generating electrical power from a heat source, such as ambient heat in blower system 100. In such embodiments, the thermopile is coupled to and provides power to UV unit 118 or other electrical components of blower system 100.

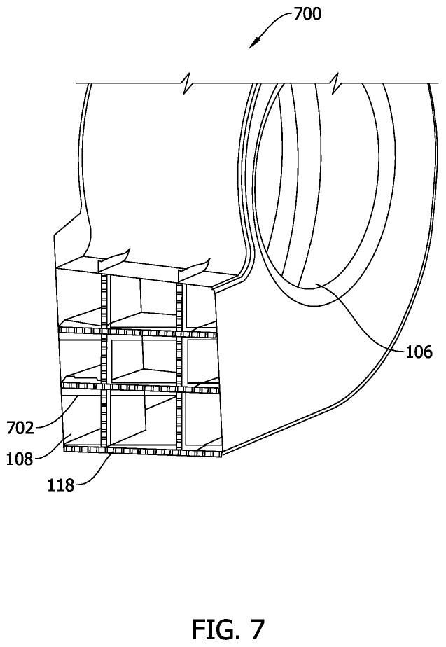

UV unit 118 is positioned within blower system 100 such that UV light emitting by UV unit 118 treats a substantial portion of, such as substantially all of, the air moving through blower system 100. In some embodiments, UV unit 118 is disposed on or near motor controller 114, or integrated into motor controller 114, such as by being at least partially disposed on or within motor controller housing 116. In such embodiments, UV unit 118 may be configured for wired communication with motor controller 114, for example, via a control line (not shown) coupled between UV unit 118 and motor controller 114. In some embodiments, UV unit 118 is disposed remotely from the motor controller 114, for example, at one or more of inlet 106 and outlet 108 of the blower system, or on baffles disposed in a flow channel of blower system 100 (as described below with respect to FIG. 7). In some such embodiments, UV unit 118 is configured for wireless communication with motor controller 114. In some embodiments, blower housing 104 includes a window, and UV unit 118 is positioned external to the blower housing 104 and adjacent to the window, such that UV radiation emitted by UV unit 118 can enter blower housing 104. In some embodiments, additional UV units 118 may be disposed in other parts of an HVAC system in which blower system 100 is installed. In some embodiments, UV unit 118 is configured to be removable from blower system 100 to ease the process of repairing or replacing the UV unit 118 should UV unit 118 fail or exceed its operational lifetime.

In some embodiments, UV unit 118 is capable of emitting short-wave UV radiation, such as (UV-C). In some such embodiments, UV unit 118 emits UV-C having a wavelength between 200 nanometers and 208 nanometers, for example, at 254 nanometers. UV-C radiation is effective at destroying pathogens but potentially dangerous to humans or components of blower system 100. In such embodiments, UV unit 118 is positioned to limit a potential for humans or delicate components to be exposed to UV-C. In some embodiments, one or more interior surfaces of blower housing 104 are reflective to the UV radiation emitted by UV unit 118, which enhances the ability of the UV radiation to treat air moving through blower system 100. Alternatively, or in addition to, one or more components of blower system 100 may be composed of a material that diffuses UV radiation such that a more-uniform pattern of UV energy is generated. In some embodiments, UV unit 118 may additionally or alternatively be configured to emit UV-A, UV-B, or other types of radiation.

As described above, in some embodiments, one or more surfaces within blower housing 104 or on blower wheel 102 are reflective to UV radiation generated by UV unit 118 or are covered by a material reflective to such UV radiation. In such embodiments, these reflective surfaces have a reflectivity greater than that of the material from which blower housing 104 or blower wheel 102 are formed. For example, in embodiments where blower housing 104 is formed from steel, which has a reflectivity of about 53% for a certain UV range emitted by UV unit 118, the reflective surfaces may include a material such as aluminum or Teflon that have a reflectivity of 78% and 95%, respectively. The material may be applied using tape, paint, or polishing, and be selected based on a desired amount of reflection, a desired cost effectiveness, availability, or other factors. In some embodiments, the reflective surfaces cover most, or substantially all, of the interior of blower housing 104 or blower wheel 102, while in some other embodiments reflective surfaces cover select parts of blower housing 104 or blower wheel 102 that enhance the transmission of UV radiation throughout blower housing 104. For example, in some such embodiments, a relatively small reflective surface is positioned immediately opposite UV unit 118. In certain embodiments, blower housing 104 or blower wheel 102 are pretreated with reflective surfaces during manufacture or before assembly, while in certain other embodiments, reflective surfaces are applied to blower housing 104 or blower wheel 102 after manufacture or assembly. In some embodiments, a use of reflective surfaces may reduce a required UV output of UV unit 118, enabling UV unit 118 to be operated at a lower power level and/or constructed using fewer LEDs 120.

In some embodiments, motor controller 114 is configured to control UV unit 118 based on an operating mode commanded by the system controller. For example, in some embodiments, motor controller 114 is configured to operate in an “OFF” mode, a heating mode, a cooling mode, a constant fan mode, and a special air treatment mode, each mode having a corresponding blower speed or airflow. In some embodiments, UV unit 118 is active only when the motor controller us operating according to the air treatment mode. In alternative embodiments, no dedicated air treatment mode is present, and UV unit 118 is active when the motor controller is operating according to one or more of the constant fan mode or the other operating modes where air moves through blower system 100. When motor controller 114 operates in the air treatment mode, the motor controller is configured to operate blower wheel 102 at a speed where the effectiveness of UV unit 118 in treating the air is relatively high, such as when the airflow rate is relatively low while still sufficient for a substantial amount of air of the space to be treated. In some embodiments, motor controller 114 includes a mechanism configured to prevent an accidental activation of UV unit 118, such as when electric motor 110 is inactive and blower wheel 102 is not moving air through blower housing 104. For example, motor controller 114 may include or be coupled to UV unit 118 via an interlock switch, door switch, connector, or software device that is activated only when motor controller is operating electric motor 110, which causes UV unit 118 to operate only when blower wheel 102 is operating and air is moving through blower housing 104.

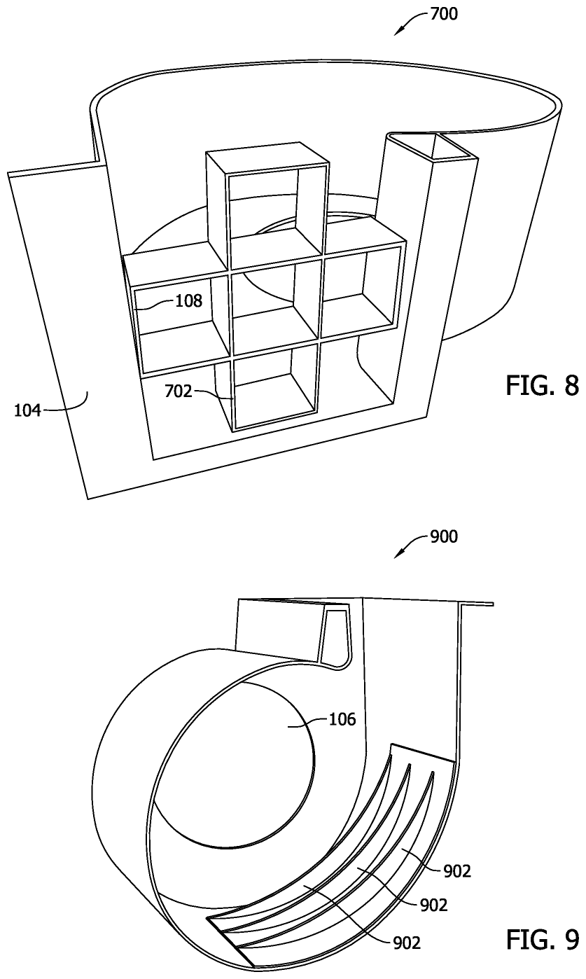

FIG. 7 is a schematic diagram of an example blower housing 700. FIG. 8 is a cross-sectional view of blower housing 700. Blower housing 700 generally functions as described with respect to blower housing 104, and includes one or more baffles 702 disposed in the airflow path between inlet 106 and outlet 108. In some embodiments, an active cleaning device such as UV unit 118 is mounted on one or more of baffles 702.

FIG. 9 is perspective diagram of another example blower housing 900. Blower housing 900 generally functions as described with respect to blower housing 104, and includes one or more baffles 902 disposed in the airflow path between inlet 106 and outlet 108. In some embodiments, an active cleaning device is mounted on one or more of baffles 902.

FIG. 10 illustrates an example electric motor 110 including an electronics module 1000. In some embodiments, electronics module 1000 is an implementation of motor controller 114 (shown in FIG. 1). Electronics module 1000 includes power electronics 1002, a processor 1004, a housing 1006, and an input/output (I/O) interface 1008. Power electronics 1002 include electrical components such as capacitors, rectifiers, and switches, which enable power electronics 1002 to convert an input power signal to provide a signal suitable for powering the stator windings of electric motor 110. Processor 1004 is coupled in communication with power electronics 1002 and configured to control switches of power electronics 1002 to generate the signal for powering the stator windings. As described with respect to motor controller 114, in some embodiments, processor 1004 is further coupled in communication with UV unit 118 and further configured to control operation of UV unit 118. In some embodiments, power electronics 1002 and processor 1004 are disposed in housing 1006. I/O interface 1008 is disposed on a wall of housing 1006, and enables external devices such as UV unit 118 or sensors to be coupled in communication with processor 1004.

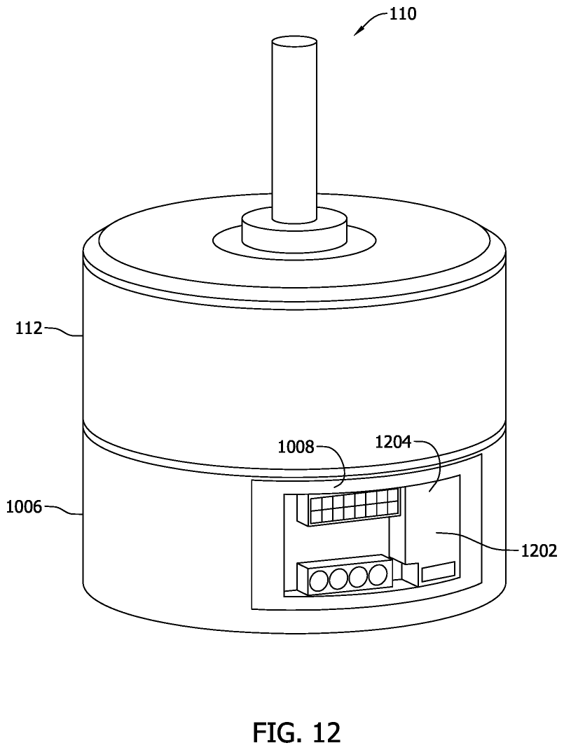

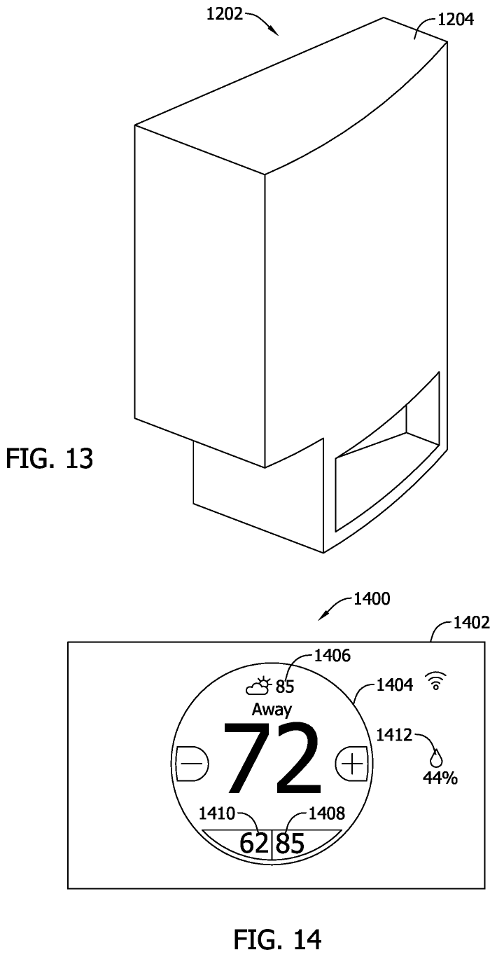

As shown in FIG. 12, a sensor module 1202 including one or more sensors may be coupled to electronics module 1000, for example, at I/O interface 1008. The sensors of sensor module 1202 are configured for detecting properties of air moving through the blower system 100, for example, to measure a quality of the moving air. The sensors may include, for example, a particulate matter sensor, volatile organic compound sensor, a temperature sensor, a humidity sensor, a carbon monoxide sensor, a carbon dioxide sensor, or other sensors. The sensors are configured for communication with processor 1004, for example, using a wired or wireless connection, such as a near field communication (NFC) connection. In some embodiments, sensor module 1202 includes a sensor housing 1204 in which one or more of the sensors are disposed. In some embodiments, sensor module 1202 is coupled to or positioned adjacent to motor controller 114. In such embodiments, sensor module 1202 may be powered by motor controller 114, for example, via a wired DC bus or via a NFC connection. Additionally or alternatively, sensor module 1202 may be powered by an internal source, such as a battery. In some embodiments, data obtained from the sensors may be displayed, for example, at the system controller or via a mobile application (“app”) executed by a user device in communication with the processor. In some embodiments, control parameters for the sensors, such as data sampling rates, are selected to achieve certain operating characteristics, such as reducing power consumption or data storage. In some embodiments, the blower system 100 may include additional sensors that generate diagnostic data, such as blockage sensors or vibration sensors, which may be disposed within or separate from sensor module 1202.

In some embodiments, blower system 100 includes sensors of any of the aforementioned types disposed separately from motor controller 114. Such sensors may be communicatively coupled to motor controller 114, UV unit 118, or a control element such as a thermostat or a local controller. This connection may be a wired connection or a wireless connection, such as a Bluetooth, Wi-Fi, or Zigbee connection. In some such embodiments, these sensors are selectively positioned to increase their respective measuring effectiveness. For example, IAQ sensors may be positioned within blower housing 104 to detect IAQ properties of air moving through blower housing 104.

In some embodiments, motor controller 114 is configured to control the speed or torque of motor 110 based on the measured quality of the air or other data obtained from sensor unit 1202 or other sensors. In some embodiments, motor controller 114 is further configured to control operation of UV unit 118 based on the measured quality of the air or other data obtained from sensor unit 1202 or other sensors.

In certain embodiments, the motor controller 114, in addition to operating the blower system in an “OFF” mode, a heating mode, a cooling mode, a constant fan mode, or a special air treatment mode, may also periodically operate the blower to cycle, or circulate, fluid in the proximity of the sensor to ensure quality measurements represent the current conditions in and around the space, and to avoid stagnant fluid, or minimal fluid flow, in the proximity of sensor module 1202. In such embodiments the period between cycles may be configurable to suit a given implementation. For example, the motor controller 114 may circulate the fluid at least every five, ten, fifteen, thirty, or more minutes. Accordingly, sensor module 1202 enables new sampling and measurement of quality, e.g., IAQ, on the selected frequency, or period.

In some embodiments, processor 1004 may control one or more of electric motor 110 or the UV unit 118 based on data received from sensor module 1202. For example, if the sensors of sensor module 1202 detect that an unhealthy level of contaminants is present in the air (e.g., a level exceeding a threshold), processor 1004 may cause the blower system 100 to operate in the air treatment mode, where the intensity of the UV light emitted by UV unit 118 is increased or the airflow is decreased. When the air quality returns to normal levels, processor 1004 is configured to cause blower system 100 to return to normal operation, such as by operating blower system 100 according to a command from the system controller. In some embodiments, the processor is configured to vary, for example, the intensity of UV light emitted by UV unit 118 or the speed of blower wheel 102 based on data received from sensor module 1202 according to one or more algorithms stored in a memory of motor controller 114. In some embodiments, if processor 1004 determines that the air quality is unhealthy, processor 1004 is configured to cause an alert message to be displayed, for example, at the system controller or via the app at the user device.

In some embodiments, the intensity of UV light emitted by UV unit 118 is adjusted based on airflow. For example, in some such embodiments, the intensity is adjusted to achieve a certain UV dosage, which is defined as the intensity multiplied by the time spent by the fluid within blower housing 104. Accordingly, a lesser airflow generally results in a greater UV dosage. The UV dosage may be selected based on a variety of factors including, for example, the volume of the space treated by blower system 100, the IAQ requirements of this space, and operating considerations of UV unit 118 such as power consumption or lifetime. In such embodiments, the airflow may be detected, for example, by motor controller 114, UV unit 118, or another control element, using an airflow sensor or pressure sensor, or a combination thereof. Based on the detected airflow, the control element determines a corresponding value, such as an intensity, a luminous flux, or an optical power, at which to operate UV unit 118 to achieve the desired dosage. This determination may be made using a lookup table or by computing the desired intensity based on the relationship between intensity, airflow, and dosage. Because the UV output of UV unit 118 may reduce over time, in some embodiments, the intensity may be adjusted over time to maintain a desired dosage. This adjustment may be determined by predicting the reduction in UV output over time, for example, based on a lookup table, or by measuring the UV output of UV unit 118 using a sensor.

In some embodiments, UV unit 118 is configured to emit UV light in response to a presence of an airflow, and cease emitting UV light in response to an absence of the airflow. In some such embodiments, UV unit 118 is coupled to motor controller 114 or a power source via a switch configured to control a supply of power to UV unit 118. The switch is actuated in response to a presence or absence of detected airflow. For example, the switch may be closed when airflow is present, and open when no airflow is present. The airflow may be detected using, for example, a pressure transducer, pressure or vacuum switch, or a temperature sensor such as a resistance temperature detector (RTD), positive temperature coefficient (PTC) or negative temperature coefficient (NTC) thermistor, or thermocouple. In addition to UV unit 118, in some embodiments, other types of active cleaning device, such as ionizers, may be configured to activate in response to the presence of the airflow.

In some embodiments, blower system 100 further includes an electrostatic filtration device configured to remove contaminants from air moving through the blower system 100. The electrostatic filtration device includes two electrodes coupled respectively to a ground and a high voltage of a DC power supply, which may be provided, for example, by motor controller 114. In some embodiments, the electrostatic filtration device is integrated into other parts of blower system 100 such as, for example, on blower wheel 102 or on individual blades of blower wheel 102. In some embodiments, the electrostatic filtration device is implemented as a mesh extending across at least a portion of the flow path of blower system 100. In some embodiments, blower system 100 or an HVAC system in which blower system 100 is installed, may further include one or more traditional air or fluid filters in addition to, or as an alternative to, the electrostatic filtration device. The traditional filters may include, for example, a fibrous or porous material capable of removing contaminants from air moving through blower system 100. In some embodiments, the electrostatic filtration device or traditional filter may be placed such that UV unit 118 may treat matter captured by the electrostatic filtration device or traditional filter, such as by killing pathogens captured in the filter. For example, the electrostatic filtration device or traditional filter may be placed at inlet 106 or outlet 108.

In some embodiments, blower system 100 further includes an ion generator. The ion generator may be disposed, for example, within blower housing 104 to treat air moving through blower housing 104. In some such embodiments, the ion generator is configured to be controlled by motor controller 116, for example, based on a detected level of contaminants or a current operating mode of blower system 100. In certain embodiments, blower system 100 may include one or more of UV unit 118, the electrostatic filtration device and the ion generator. For example, in some such embodiments, the ion generator may be used in conjunction with or without the electrostatic filtration device, and no UV unit 118 is present.

In some embodiments, electric motor 110, motor controller 114, and one or more of sensor module 1202, UV unit 118, or other components of blower system 100 may be integrated into a single motor package. Accordingly, a legacy blower system can be upgraded to include air quality control capabilities by replacing an original equipment manufacturer (OEM) motor of the legacy blower system with the single motor package.

FIG. 14 depicts an exemplary user interface 1400. In some embodiments, processor 1004 is configured to cause user interface 1400 to be displayed, for example, via an app or web page displayed on a user device. As illustrated in FIG. 14, in some embodiments, user interface 1400 includes a virtual thermostat 1402 that enables a user to, for example, view a current temperature setting 1404 and to adjust the current temperature setting. In some embodiments, user interface 1400 further includes information about current temperature or weather conditions such as, for example, a current outdoor temperature 1406, a daily high temperature 1408, a daily low temperature 1410, and a chance of precipitation 1412. In some embodiments, the current temperature and weather conditions are retrieved by processor 1004 via the Internet.

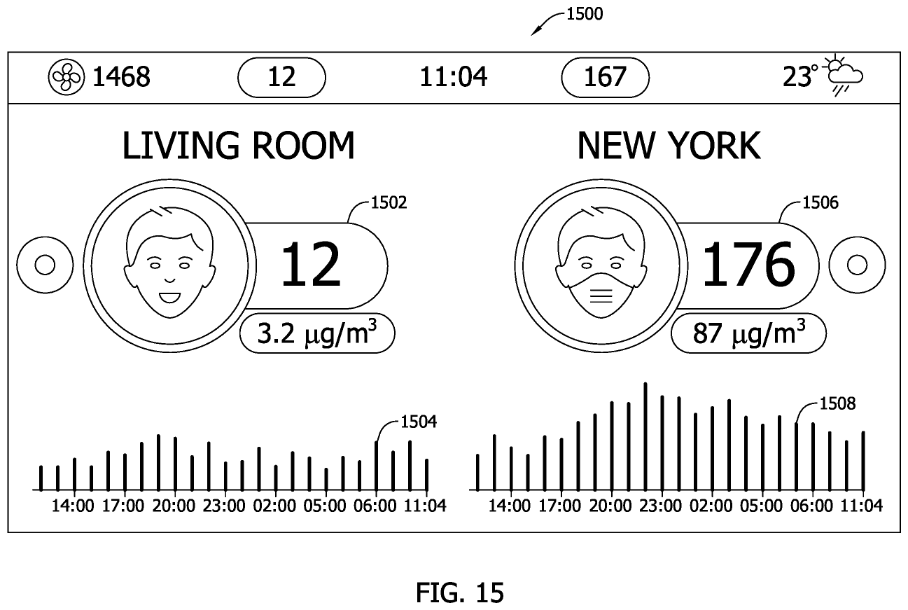

FIG. 15 depicts another exemplary user interface 1500. Like user interface 1400, in some embodiments, processor 1004 is configured to cause user interface 1500 to be displayed, for example, via an app or web page displayed on a user device. As illustrated in FIG. 15, in some embodiments, user interface 1500 is configured to include air quality data, such as particulate matter data, for different times at multiple locations. For example, in some embodiments, user interface 1500 includes a current indoor particulate matter level 1502, historical indoor particulate matter levels 1504, a current outdoor particulate matter level 1506, and historical outdoor particulate matter levels 1508. In some such embodiments, the user interface further includes an indicator of current air quality, for example, whether the current air quality is health or unhealthy.

In some embodiments, processor 1004 is configured to generate alerts, notifications, reminders, or warnings relating to maintenance of blower system 100. These alerts may correspond to, for example, a need to clean or replace LEDs 120 of UV unit 118, or an indication that UV unit 118 is not functioning as intended. Alerts corresponding to periodic maintenance, such as cleaning or replacing LEDs 120, may be generated periodically to coincide with recommended periods. For example, an alert to clean LEDs 120 may be generated once each six months. In some embodiments, a power consumption of UV unit 118 and/or a time that UV unit 118 is operating is tracked, and an alert is generated once the tracked power consumption and/or operating time exceeds a threshold. Alerts corresponding to UV unit 118 not functioning as intended may be generated in response to factors such as a detection of, for example, a lack of UV light when UV unit 118 is active, a measured temperature of UV unit 118 falling outside a threshold range, or an IAQ falling outside of a desired range during operation of blower system 100. These factors may be detected using sensors, such as those of sensor unit 1202 or other sensors located within blower system 100 or a space treated by blower system 100. Examples of alerts include icons, messages, or sounds generated by user interface 1400 or user interface 1500, indicator lights located on one or more components of blower system 100, or audible alerts generated by one or more components of blower system 100. Such alerts may be generated at a frequency selected to be noticeable to a user without undue annoyance. Such a frequency may depend on a seriousness of the alert. For example, an audible alert indicating an unsafe IAQ, such as a presence of carbon monoxide, may run continuously, while an audible alert corresponding to a suggestion for periodic cleaning may repeat once per day.

In some embodiments, blower system 100 includes one or more mechanisms to ensure UV unit 118 is deactivated before maintenance is performed on UV unit 118, so that any person performing maintenance on UV unit 118 is not exposed to potentially harmful UV radiation, such as UV-C radiation. In some such embodiments, blower system 100 includes an indicator light that illuminates in response to UV unit 118 being active or inactive. For example, the indicator light may illuminate when UV unit 118 is active and it is unsafe to open blower housing 104 or to remove UV unit 118, or UV 118 may illuminate when UV unit 118 is inactive and it is safe to open blower housing and to remove UV unit 118. The indicator light may be labeled accordingly, and located where it can be easily viewed by a person performing maintenance on blower system 100. The indicator light may illuminate in response to a control signal that controls activation of UV unit 118, or may illuminate in response to a detection of UV light within blower housing 104. For example, the indicator light may be controlled based on the output of an optical sensor located within blower housing 104, which is some examples may be configured to detect UV light emitted by UV unit 118. In certain embodiments, a photoluminescent strip is located within blower housing 104 and is configured to emit light or change color in response to UV light emitted by UV unit 118. The photoluminescent strip may serve as a visual indicator that UV unit 118 is active, or may be read by an optical sensor that in turn controls, for example, an illumination of the indicator light. In some embodiments, UV unit 118 includes a switch, such as a mechanical switch, a magnetic switch, or a proximity switch, from which UV unit 118 can be deactivated manually. In some such embodiments, the mechanical switch is attached to a door or openable access point of blower housing 104, such that when the door of openable access point is opened, UV unit 118 is automatically deactivated. In some embodiments, UV unit 118 is configured to be activatable only when installed in a proper configuration or suitable enclosure, such as one preventing persons from being exposed to UV light. For example, UV unit 118 may include a proximity sensor capable of detecting whether UV unit 118 has been installed in a suitable enclosure, or UV unit 118 may include a physical switch, a magnetic switch, or a proximity switch that close upon installation. In some such embodiments, airflow detection may also be used to determine that UV unit 118 has been installed safely.

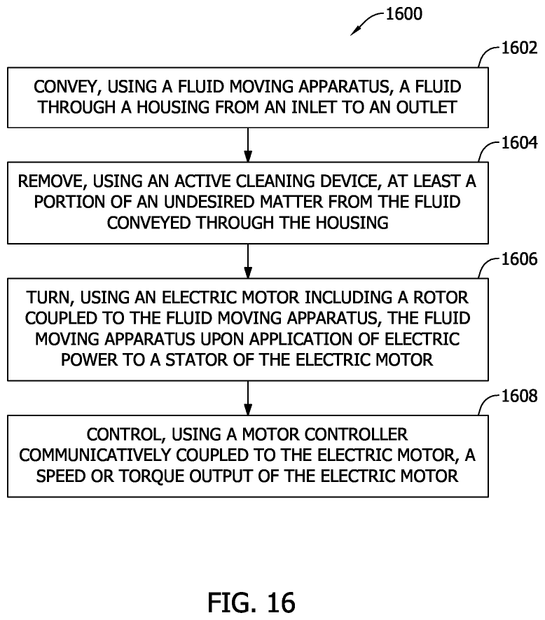

FIG. 16 illustrates an exemplary method 1600 for operating blower system 100. Blower system 100 conveys 1602, using blower wheel 102, a fluid through blower housing 104 from inlet 106 to outlet 108. Blower system 100 also removes 1604, using UV unit 118, at least a portion of an undesired matter from the fluid conveyed through blower housing 104. Blower system also 100 turns 1606, using electric motor 110 including a rotor coupled to blower wheel 102, blower wheel 102 upon application of electric power to a stator of electric motor 110. Blower system 100 also controls 1608, using motor controller 114 communicatively coupled to electric motor 110, at least one of a speed output or a torque output of electric motor 110.

In some embodiments, blower system 100 is configured to self-clean UV unit 118. For example, UV unit 118 may be shaped or positioned such that a fluid flow reduces an accumulation of or removes dust accumulated on LEDs 120. In such embodiments, blower system 100 may be operated in a speed burst mode, in which a speed of blower wheel 102 is increased, generating an increased fluid flow to clean UV unit 118. Blower system 100 may be operated in the speed burst mode periodically, intermittently, or in response to a detected accumulation of matter at UV unit 118 that reduces its UV output. The accumulation may be detected, for example, by detecting a reduction in UV light emitted by UV unit 118. In certain embodiments, UV unit 118 includes a cover configured to protect LEDs 120 from dust. The cover may be transparent to the UV light emitted by LEDs 120.

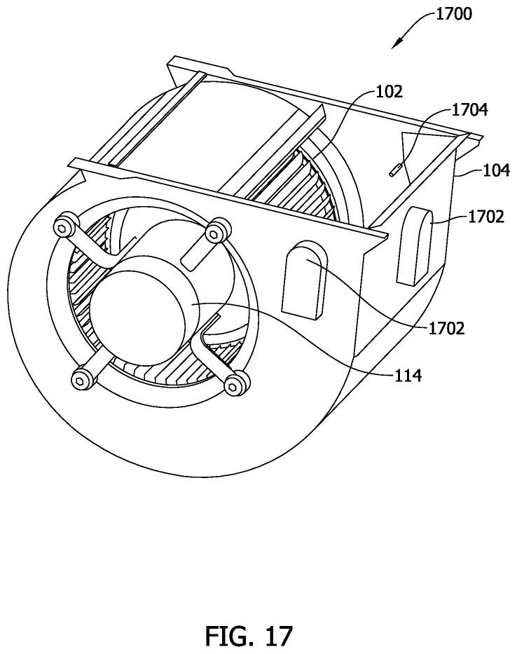

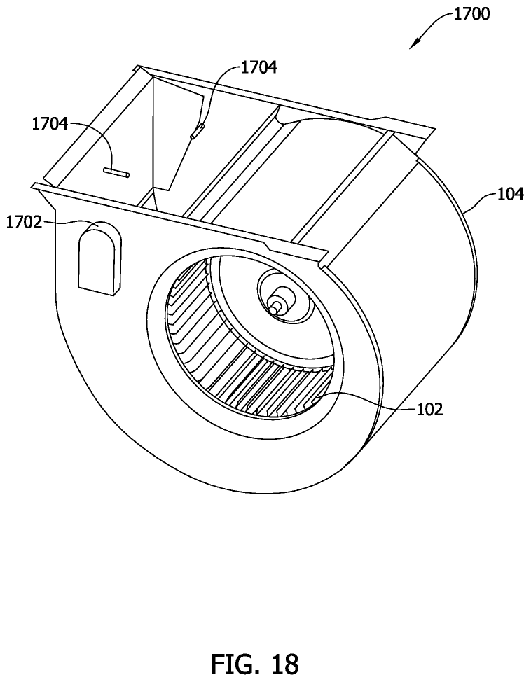

FIGS. 17 and 18 depict another exemplary blower system 1700. Blower system 1700 includes ionizers 1702 mounted on blower housing 104. Each ionizer 1702 includes one or more probes 1704 extending into an interior of blower housing 104. When active, probes 1704 are electrically charged to create ions within a fluid moving through blower housing 104, which interact with particles to remove the particles from the fluid flow. In some embodiments, as shown in FIGS. 17 and 18, ionizers 1702 are positioned at an outlet of blower housing 104 to increase an amount of fluid that is treated by ionizers 1702. Additionally or alternatively, ionizers 1702 may be positioned at a different location within blower housing 104, on blower wheel 102, or on a duct or other fluid conduit coupled to blower system 1700. In some embodiments, ionizers 1702 are integrated with UV unit 118 as a single package.

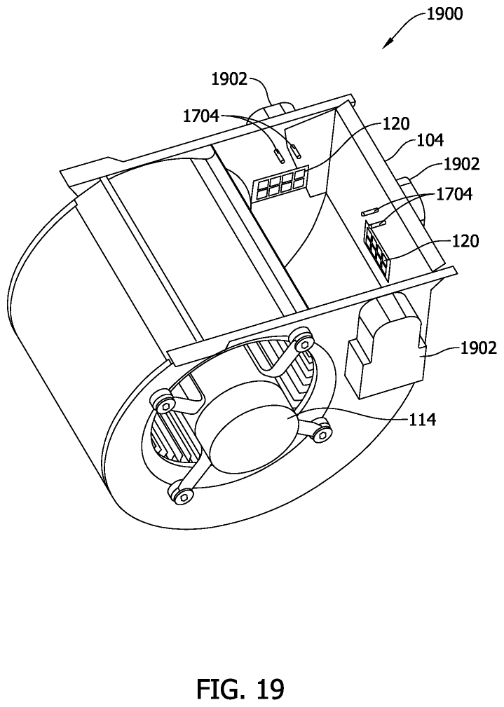

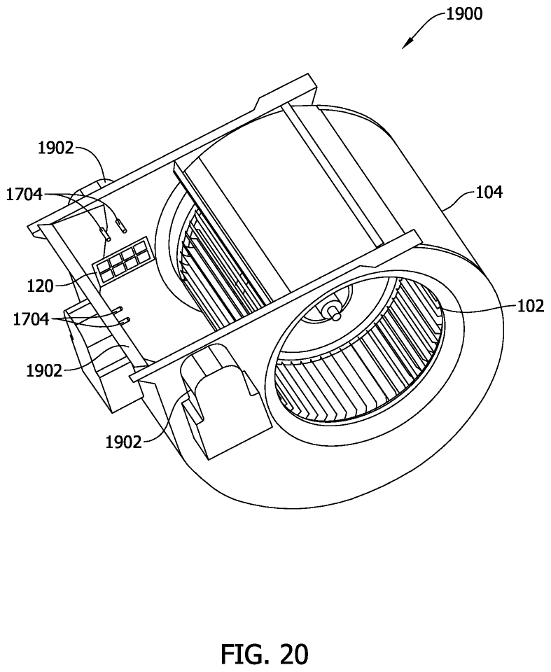

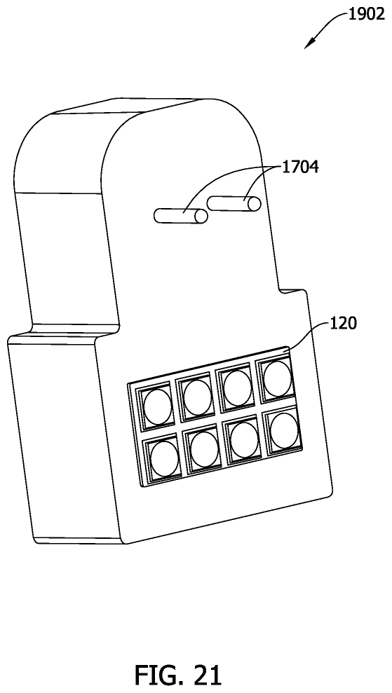

FIGS. 19 and 20 depict another exemplary blower system 1900. Blower system 1900 includes ionizer and UV combination units 1902 mounted on blower housing 104. As shown in FIG. 21, ionizer and UV combination unit 1902 includes both UV LEDs 120 (described with respect to FIG. 1) and ionizer probes 1702, so that ionizer and UV combination unit 1902 serves as a combination of UV unit 118 and ionizer 1702. Accordingly, both LEDs 120 and probes 1704 may share a power supply and control. In some embodiments, in addition or alternatively to being mounted on blower housing 104, ionizer and UV combination unit 1902 is mounted at another location, such as a duct. In some embodiments, additional active cleaning devices, such as an electrostatic filter, are integrated into ionizer and UV combination units 1902 and/or share a power supply and control with LEDs 120 and probes 1704.

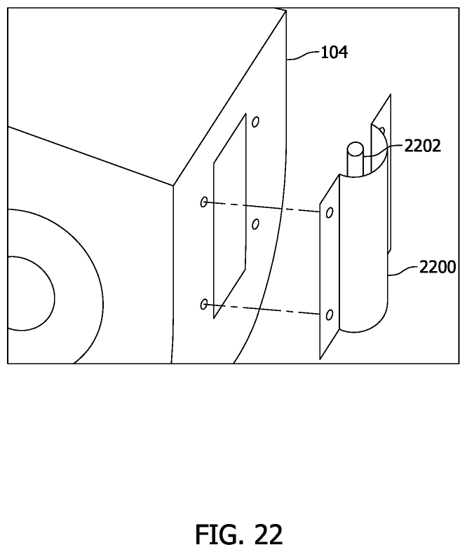

FIG. 22 depicts an exemplary UV lamp 2200. UV lamp 2200 may be incorporated into UV unit 118 or be separate from UV unit 118. For example, UV unit 118 may include the UV lamp 2200 in addition to or as an alternative to LEDs 120. UV unit includes a UV emitting element 2202 that emits UV light to treat fluid flowing through, for example, blower system 100. In some embodiments, UV emitting element 2202 is a mercury-based discharge lamp or other lamp capable of emitting UV light. In such embodiments, UV emitting elements 2202 may generate UV light of a greater intensity than that generated by, for example, LEDs 120. Because such lamps may take longer to transition between an active and inactive state, UV lamp 2200 may be operated for longer periods of time, such as one or two hours, and transaction between the inactive to active states less frequently than, for example, UV unit 118. In certain embodiments, UV emitting elements 2202 emit UV light at a wavelength of approximately 254 nanometers. As shown in FIG. 19, UV lamp 2200 may be positioned on blower housing 104 to treat fluid passing therethrough. Alternatively, UV lamp 2200 may be positioned on another part of an HVAC system coupled to blower system 100, such as a duct or proximate to a heating or cooling coil, or installed as a part of a stand-alone air scrubbing unit not coupled to an HVAC system.

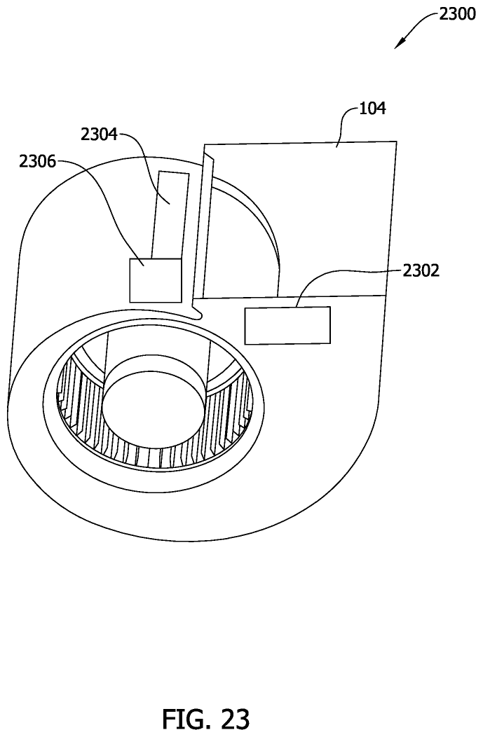

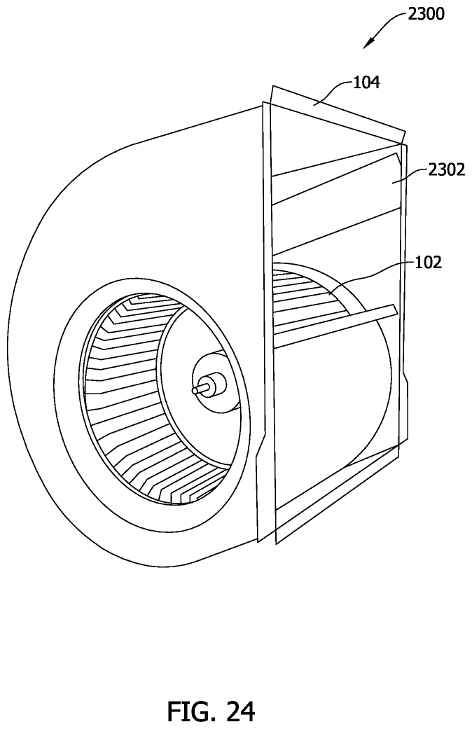

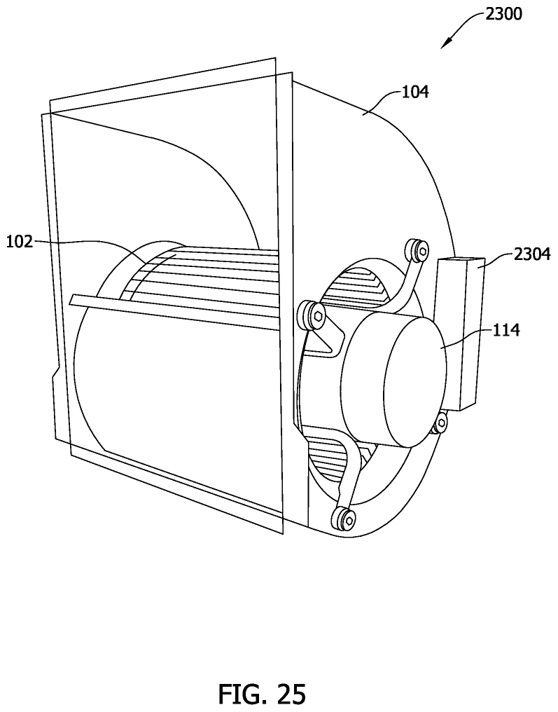

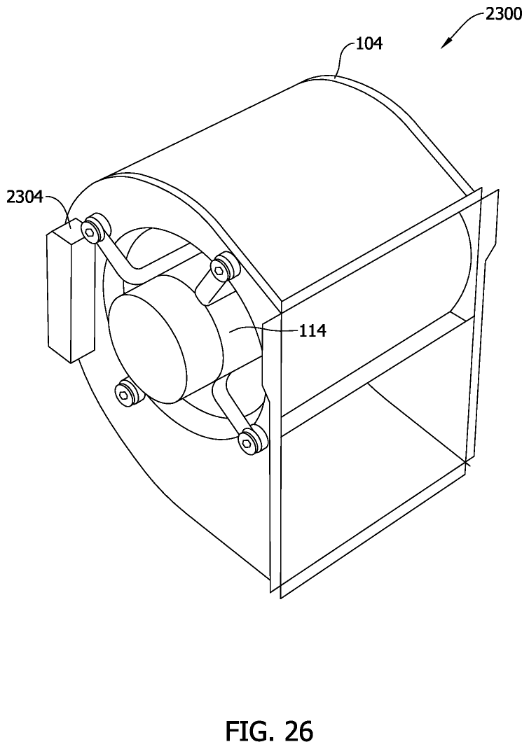

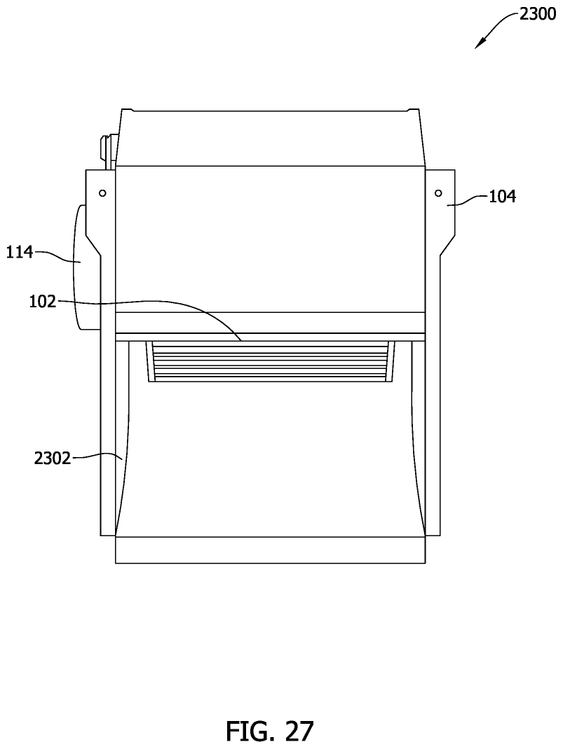

FIGS. 23-27 depict a modular air treatment system 2300. Modular air treatment system 2300 includes a source emitter such as UV emitter 2302, a power supply 2304, and a control unit 2306. In the embodiment depicted in FIG. 20, UV emitter 2302, power supply 2304, and control unit 2306 are disposed on blower housing in 104 described with respect to FIG. 1. In other embodiments, UV emitter 2302, power supply 2304, and control unit 2306 are disposed at another location, such as a motor frame of electric motor 110, a portion of an HVAC system separate from the blower, or a stand-alone unit not part of an HVAC system. In such embodiments, the system in which modular air treatment system 2300 is installed may not necessarily include a blower housing such as blower housing 104. In some embodiments, at least some of UV emitter 2302, power supply 2304, and control unit 2306 are integrated into a single unit and/or disposed within a single housing. For example, power supply 2304 and control unit 2306 may be implemented on a single circuit board. In the embodiment depicted in FIG. 20, one or more of UV emitter 2302, power supply 2304, and control unit 2306 are mounted on blower housing 104 using screws, magnets, adhesives, or another attaching device.

UV emitter 2302 is configured to emit UV light, such as UV-C light, to treat air moving through blower unit 104. UV emitter 2302 includes a UV light source such as, for example UV LEDs or one or more UV lamps. In some embodiments, UV emitter 2302 generally functions as described with respect to UV unit 118 (shown in FIG. 1). In some embodiments, UV emitter 2302 is shaped and/or positioned to decrease or minimize an airflow impedance or to increase or maximize a UV dosage. UV emitter 2302 is supplied power by power supply 2304. For example, power supply 2304 may produce a supply of 12-volt or 24-volt DC power to power UV emitter 2302. In some embodiments, modular air treatment system 2300 may include another type of active cleaning device, such as an ionizer or electrostatic filter, in addition to or alternatively to UV emitter 2302.

Control unit 2306 is configured to control activation of UV emitter 2302 and/or other active cleaning devices of modular air treatment system 2300 by controlling the supply of power of UV emitter 2302 and/or the other active cleaning devices. For example, control unit 2306 may include relays configured to couple and decouple UV emitter 2302 from power supply 2304. In some embodiments, control unit 2306 is configured to couple UV emitter 2302 to power supply 2304 to activate UV emitter 2302 when the blower is active, and to decouple UV emitter 2302 from power supply 2304 to deactivate UV emitter 2302 when the blower is inactive. In such embodiments, control unit 2306 is configured to determine when the blower is active.

In certain embodiments, control unit 2306 is configured to determine when the blower is active and an airflow is present by detecting a control signal for the blower. For example, in some embodiments, the blower is controlled by a thermostat using one or more 24-volt AC analog signals. In such embodiments, control unit 2306 is coupled in communication with the thermostat and configured to receive the control signals along with the blower. In such embodiments, control unit 2306 activates UV emitter 2302 and/or another active cleaning device in response to receiving the control signal, and deactivates UV emitter 2302 in response to an absence of the control signal. For example, in some embodiments, the control signals are transmitted via green (G), white (W), and yellow (Y) 24 volt AC control lines, and control unit 2306 is configured to activate UV emitter 2302 if a signal is present on any of the three lines. Accordingly, in such embodiments, UV emitter 2302 is active when the blower is operating and air is moving though blower housing 102, and is inactive when no airflow is present.

In some embodiments, control unit 2306 is configured to determine when the blower is active and a fluid flow is present by detecting movement of the blower or the fluid flow itself. In some such embodiments, control unit 2306 is configured to sense fluid velocity using a thermistor in self-heat mode. The thermistor may be mounted, for example, on side walls of blower housing 104 where interference with airflow is reduced. In certain embodiments, control unit 2306 is coupled to a radio frequency (RF) proximity detection chip mounted near the blower and is configured to determine the speed of the blower based on a signal output by the RF proximity detection chip corresponding to the position of blower blades. Utilizing the RF proximity detection chip to measure the blower speed reduces a likelihood that the measurement is influenced by a presence of dust. In some embodiments, control unit 2306 is coupled to a pressure sensor mounted near the blower and is configured to determine that the blower is operating based on air or fluid pressure waves resulting from blades of the blower passing. In some embodiments, in addition to determining to activate or deactivate UV emitter 2302 based on data received from such sensors, control unit 2306 is configured to determine, for example, a power or intensity at which to operate UV emitter 2302 based on measurements received from the sensors. In certain embodiments, control unit 2306 is configured to communicate with a blower control, such as motor controller 114 or a thermostat, using NFC, Bluetooth, or other wireless or wired communication to determine whether the blower is active and/or determine the current blower speed or airflow level.

In some embodiments, modular air treatment system 2300 further includes a status indicator that visibly and/or audibly indicates whether UV emitter 2302 is active or inactive. The status indicator may be, for example, an LED. In some embodiments, the LED is disposed on a same circuit board as control unit 2306 and is visible outside of blower housing 104 via a clear mounting structure.

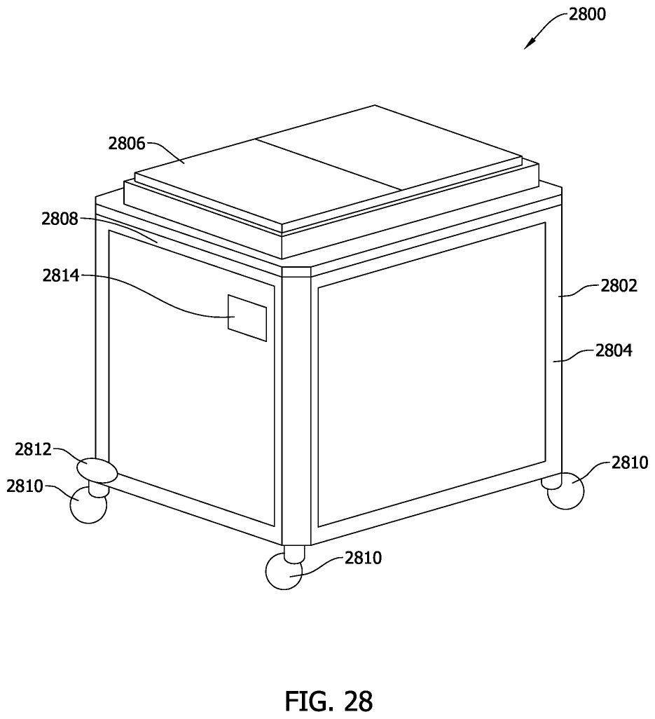

FIG. 28 illustrates an exemplary scrubber unit 2800. Scrubber unit may be implemented using, for example, air treatment system 2300 to provide a portable device for treating IAQ. For example, air treatment system 2300 may be contained within scrubber unit 2800. Scrubber unit 2800 includes a housing 2802, an inlet 2804 through which air enters housing 2802, and an outlet though which treated air exits housing 2802. In some embodiments, housing 2802 is formed from galvanized steel panels or another metal, ceramic, plastic, or composite material. In certain embodiments, outlet 2806 includes a double deflection grill to increase airflow discharge. In some embodiments, housing 2802 is insulated with a material such as fiberglass.

In some embodiments, scrubber unit 2800 further includes a handle 2808 or wheels 2810 to increase portability of scrubber unit 2800. In certain embodiments, scrubber unit 2800 includes a power connector 2812 through which scrubber unit 2800 receives a power signal, such as line power, to power air treatment system 2300. In some embodiments, scrubber unit 2800 further includes a control panel 2814 coupled in communication with and configured to control air treatment system 2300. In some such embodiments, control panel 2814 is a display screen configured to display, for example, user interface 1400 or user interface 1500. The display may include diagnostic data such as life left on a filter or UV emitter 2302 and a cleaning status. In some embodiments, scrubber unit 2800 further includes a filter, such as a minimum efficiency reporting value (MERV) 8 filter, through which air moving through housing 2802 may pass to further treat the moving air and/or a post filter located at a blower discharge to trap smaller particles. In some embodiments, UV emitter 2302 consists of a number of lamps, and radiation produced by UV emitter 2302 is fully contained within housing 2802 so no radiation is released outside scrubber unit 2800.

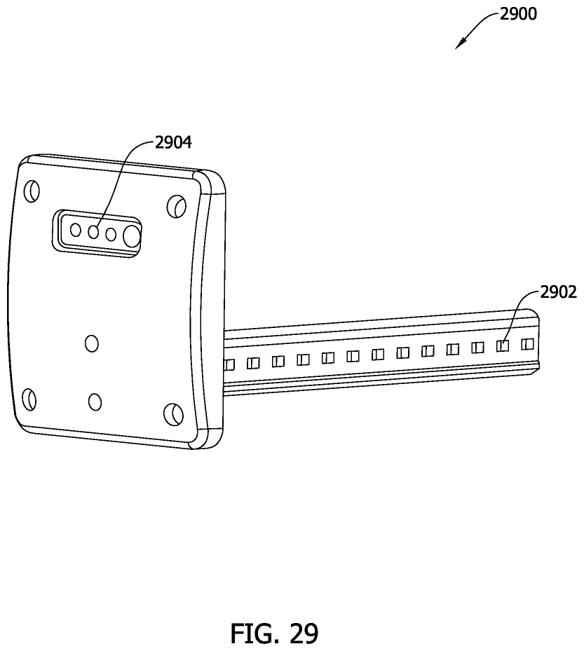

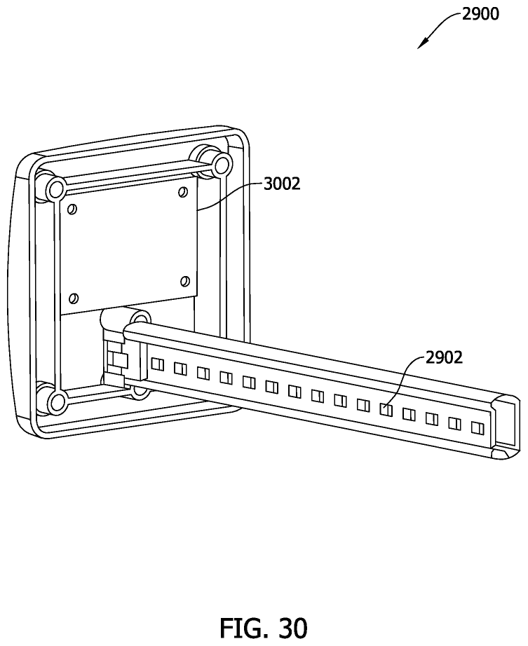

FIGS. 29 and 30 illustrate an example UV emitter 2900. In some embodiments, UV emitter 2900 is similar to UV emitter 2302. UV emitter 2900 includes UV elements 2902, indicator lights 2904, and a printed circuit board (PCB) 3002. UV elements 2902 are configured to emit UV-C light, and generally function as described with respect to LEDs 120. Indicator lights 2904 are configured to be illuminated to indicate a status of UV emitter 2900, for example, any combination of an active or inactive state, a presence of a fault, cleaning or maintenance due, and/or another status. PCB 3002 includes circuitry configured to control operation of UV elements 2902 and indicator lights 2004, such as a controller, a power supply, and/or sensors configured to detect a presence of an airflow and/or other parameters.

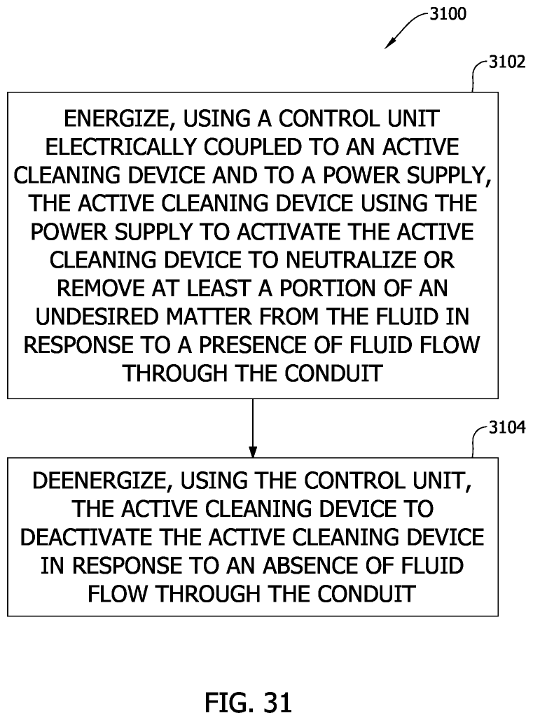

FIG. 31 illustrates an exemplary method 3100 for using air treatment system 2300 to treat a fluid, such as air, moving though a conduit, such as blower housing 104. Control unit 2306 energizes 3102 UV emitter 2302 or another active cleaning device using power supply 2304 to activate UV emitter 2302 to neutralize or remove at least a portion of an undesired matter from a fluid in response to a presence of fluid flow through the conduit. Control unit 2306 deenergizes 3104 UV emitter 2302 or the other active cleaning device to deactivate UV emitter 2302 in response to an absence of fluid flow through the conduit.

In some embodiments, UV emitter 2302 includes a UV LED or a mercury-based discharge lamp. In certain embodiments, UV emitter 2302 is configured to emit UV-C radiation. In some embodiments, UV emitter 2302 is shaped to reduce an accumulation of matter on UV emitter 2302 from the fluid flow. In certain embodiments, at least one of UV emitter 2302, power supply 2304, or control unit 2306 is disposed on PCB 3002.

In some embodiments control unit 2306 measures a fluid flow through the conduit, computes at least one of an intensity, a luminous flux, or an optical power at which to operate UV emitter 2302, and operates UV emitter 2302 at the computed intensity, luminous flux, or optical power.

In some embodiments, wherein air treatment system 2300 is incorporated into a blower system such as blower system 100, the blower system is controlled by a thermostat. In some such embodiments, control unit 2306 is coupled in communication with the thermostat and configured to determine the fluid flow is present based on a presence of a control signal generated by the thermostat. In certain embodiments, control unit 2306 additionally or alternatively determines the fluid flow is present based on a detection of movement of a blower wheel, such as blower wheel 102. In some embodiments, control unit 2306 additionally or alternatively determines the fluid flow is present based on a detection of fluid flow through the blower housing. In certain embodiments, at least one of UV emitter 2302, power supply 2304, control unit 2306, or another active cleaning device is disposed on the blower housing.

In some embodiments, a status indicator is configured to generate at least one of light or sound in response to an activation of UV emitter 2302. In some such embodiments, the status indicator includes an LED.

In some embodiments, a reflective material is disposed in the conduit and configured to distribute UV radiation emitted by the UV emitter. In some such embodiments, the reflective material includes aluminum, Teflon, or another material with a relatively high reflectivity. The reflective material may be adhered to blower housing 104 or applied as a paint or polishing.

In some embodiments, control unit 2306 detects a failure of UV emitter 2302 and/or another active cleaning device of air treatment system 2300 and generates a visible alert or an audible alert indicating the active cleaning device has failed or its intensity is below a predefined level. In some such embodiments, to detect a failure of UV emitter 2302, control unit 2306 is configured to determine a measured parameter falls outside of a threshold range. For example, control unit 2306 may detect that no or a reduced amount of UV light is present when UV emitter 2302 is receiving power, and may accordingly determine that UV emitter 2302 has failed. In some such embodiments, control unit 2306 additionally determines there is a need for service or cleaning.

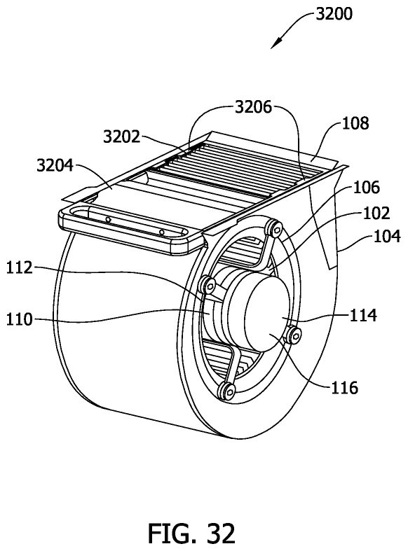

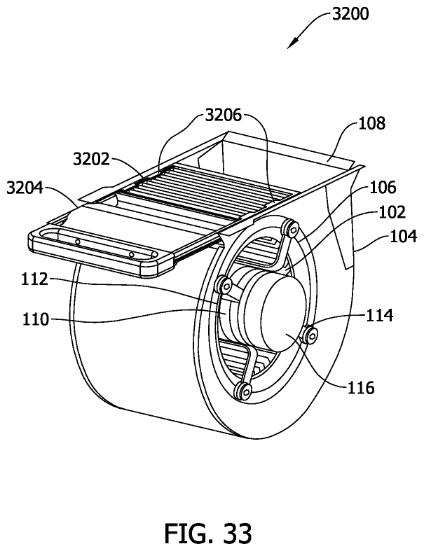

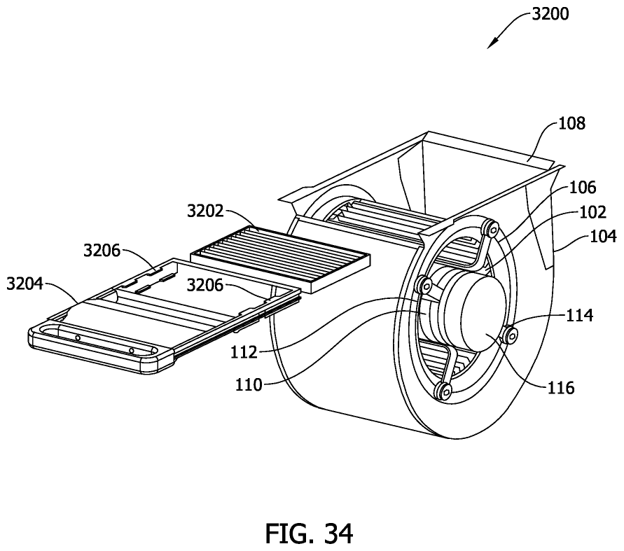

FIGS. 32, 33, and 34 illustrate an exemplary blower system 3200. Blower system 3200 includes blower wheel 102, blower housing 104, motor 110, motor housing 112, motor controller 114, and motor controller housing 116, which generally function as described with respect to FIG. 1. In some embodiments, blower system 3200 further includes an active cleaning system such as air treatment system 2300. Blower system 3200 further includes a filter 3202 and a filter tray 3204. Filter 3202 and filter tray 3204 are disposed upon outlet 108 of blower housing 104, such that air moving through blower housing 102 passes through filter 3202. When positioned at outlet 108, filter 3202 helps capture harmful particles in a space reachable by UV treatment within the blower. Alternatively, filter 3202 and filter tray 3204 may be disposed at another location within blower housing 104 where air may move through filter 3202. In some embodiments, filter tray 3204 is removably attached to blower housing 104. For example, as described in further detail below, filter tray 3204 may be slid into a position where filter tray 3204 is held in place with respect to blower housing 104 by a receiving mechanism. In some embodiments, filter 3202 is removably attached to filter tray 3204. In some embodiments, such as those wherein blower system 3200 is installed into an HVAC system, filter 3202 and filter tray 3204 can be detached and removed from blower housing 104 without removing blower system 3200 from the HVAC system.

Filter 3202 is configured to capture particles moving through outlet 108. Filter 3202 may include, for example, a fibrous or porous material that allows fluid to pass through while capturing particles. In some embodiments, filter 3202 is a secondary filter of an HVAC system. In other words, the HVAC system in which filter 3202 is installed may include a primary filter in flow communication with blower system 3200 configured to capture, for example, larger particulate matter such as dust. In such embodiments, filter 3202 may be configured to capture smaller particulate matter, such as pathogens. Because the amount of particles reaching filter 3202 is reduced in such a configuration, by using filter 3202 in conjunction with a primary filter, filter 3202 may require cleaning or replacement less frequently. In certain embodiments, filter 3302 is positioned in proximity to an active cleaning device, enabling further deactivation of active bacteria/viruses caught in filter 3202 for safer handling of filter 3202 during replacement. In some embodiments, filter 3202 includes an electrostatic filter.

Filter tray 3204 is configured to hold filter 3202 in position with respect to outlet 108 of motor housing 104. For example, filter 3202 may be slid into a position within filter tray 3204 where, when filter tray 3204 is installed within blower housing 104, filter 3202 is held in place with respect to blower housing 104. In some embodiments, to enable filter tray 3204 to be installed within blower housing 104, filter tray 3204 includes tracks 3206 and is further configured to slide into position with respect to motor housing 104 along tracks 3206. In some such embodiments, blower housing 104 includes lances disposed along an edge of outlet 108 that are configured to receive tracks 3206 of filter tray 3204 to hold filter tray 3204 in position. Additionally or alternatively, motor housing 104 may include other features for securing filter tray 3204 in place. For example, in some embodiments, filter 3202 or filter tray 3204 may be fastened to blower housing 104 using screws or another locking mechanism.

In the foregoing specification and the claims that follow, a number of terms are referenced that have the following meanings.

As used herein, an element or step recited in the singular and preceded with the word “a” or “an” should be understood as not excluding plural elements or steps, unless such exclusion is explicitly recited. Furthermore, references to “example implementation” or “one implementation” of the present disclosure are not intended to be interpreted as excluding the existence of additional implementations that also incorporate the recited features.

“Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here, and throughout the specification and claims, range limitations may be combined or interchanged. Such ranges are identified and include all the sub-ranges contained therein unless context or language indicates otherwise.

Disjunctive language such as the phrase “at least one of X, Y, or Z,” unless specifically stated otherwise, is generally understood within the context as used to state that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present. Additionally, conjunctive language such as the phrase “at least one of X, Y, and Z,” unless specifically stated otherwise, should also be understood to mean X, Y, Z, or any combination thereof, including “X, Y, and/or Z.”

Some embodiments involve the use of one or more electronic processing or computing devices. As used herein, the terms “processor” and “computer” and related terms, e.g., “processing device,” “computing device,” and “controller” are not limited to just those integrated circuits referred to in the art as a computer, but broadly refers to a processor, a processing device, a controller, a general purpose central processing unit (CPU), a graphics processing unit (GPU), a microcontroller, a microcomputer, a programmable logic controller (PLC), a reduced instruction set computer (RISC) processor, a field programmable gate array (FPGA), a digital signal processing (DSP) device, an application specific integrated circuit (ASIC), and other programmable circuits or processing devices capable of executing the functions described herein, and these terms are used interchangeably herein. The above embodiments are examples only, and thus are not intended to limit in any way the definition or meaning of the terms processor, processing device, and related terms.

In the embodiments described herein, memory may include, but is not limited to, a non-transitory computer-readable medium, such as flash memory, a random access memory (RAM), read-only memory (ROM), erasable programmable read-only memory (EPROM), electrically erasable programmable read-only memory (EEPROM), and non-volatile RAM (NVRAM). As used herein, the term “non-transitory computer-readable media” is intended to be representative of any tangible, computer-readable media, including, without limitation, non-transitory computer storage devices, including, without limitation, volatile and non-volatile media, and removable and non-removable media such as a firmware, physical and virtual storage, CD-ROMs, DVDs, and any other digital source such as a network or the Internet, as well as yet to be developed digital means, with the sole exception being a transitory, propagating signal. Alternatively, a floppy disk, a compact disc-read only memory (CD-ROM), a magneto-optical disk (MOD), a digital versatile disc (DVD), or any other computer-based device implemented in any method or technology for short-term and long-term storage of information, such as, computer-readable instructions, data structures, program modules and sub-modules, or other data may also be used. Therefore, the methods described herein may be encoded as executable instructions, e.g., “software” and “firmware,” embodied in a non-transitory computer-readable medium. Further, as used herein, the terms “software” and “firmware” are interchangeable, and include any computer program stored in memory for execution by personal computers, workstations, clients and servers. Such instructions, when executed by a processor, cause the processor to perform at least a portion of the methods described herein.

Also, in the embodiments described herein, additional input channels may be, but are not limited to, computer peripherals associated with an operator interface such as a mouse and a keyboard. Alternatively, other computer peripherals may also be used that may include, for example, but not be limited to, a scanner. Furthermore, in the exemplary embodiment, additional output channels may include, but not be limited to, an operator interface monitor.

The systems and methods described herein are not limited to the specific embodiments described herein, but rather, components of the systems and/or steps of the methods may be utilized independently and separately from other components and/or steps described herein.

Although specific features of various embodiments of the disclosure may be shown in some drawings and not in others, this is for convenience only. In accordance with the principles of the disclosure, any feature of a drawing may be referenced and/or claimed in combination with any feature of any other drawing.

This written description uses examples to provide details on the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A fluid treatment system for treating a fluid moving through a conduit, said fluid treatment system comprising:
an active cleaning device configured to neutralize or remove at least a portion of an undesired matter in the fluid;
a power supply configured to supply electric power to said active cleaning device; and
a control unit electrically coupled to said active cleaning device and to said power supply, said control unit configured to:
energize said active cleaning device using said power supply to activate said active cleaning device in response to a presence of fluid flow through the conduit; and
deenergize said active cleaning device to deactivate said active cleaning device in response to an absence of fluid flow through the conduit.

2. The fluid treatment system of claim 1, wherein said active cleaning device comprises an ultraviolet (UV) emitter, said UV emitter comprising at least one of a UV light emitting diode (LED) or a mercury-based discharge lamp, said UV emitter configured to emit UV-C radiation.

3. The fluid treatment system of claim 2, wherein said control unit is further configure to:
measure a fluid flow through the conduit;
compute at least one of an intensity, a luminous flux, or an optical power at which to operate said UV emitter based on the fluid flow; and operate said UV emitter at the computed intensity, luminous flux, or optical power.

4. The fluid treatment system of claim 1, wherein said active cleaning device comprises a UV emitter and an ionizer integrated into a single package.

5. The fluid treatment system of claim 1, wherein at least one said active cleaning device, said power supply, or said control unit is disposed on a printed circuit board (PCB).

6. The fluid treatment system of claim 1, wherein said active cleaning device is shaped to reduce an accumulation of matter on said active cleaning device from the fluid flow.

7. The fluid treatment system of claim 1, wherein said active cleaning device comprises an ionizer.

8. The fluid treatment system of claim 1, wherein the conduit includes a blower housing of a blower system.

9. The fluid treatment system of claim 8, wherein said control unit is configured to determine the fluid flow is present based on a detection of fluid flow through the blower housing.

10. The fluid treatment system of claim 8, wherein at least one of said active cleaning device, said power supply, or said control unit is disposed on the blower housing.

11. The fluid treatment system of claim 1, wherein said control unit is coupled in communication with a thermostat and is configured to determine the fluid flow is present based on a presence of a control signal generated by the thermostat.

12. The fluid treatment system of claim 1, wherein said control unit is configured to determine the fluid flow is present based on a detection of movement of a blower wheel of a blower system coupled to the conduit.

13. The fluid treatment system of claim 1, further comprising a status indicator configured to generate at least one of light or sound in response to an activation of said active cleaning device.

14. The fluid treatment system of claim 13, wherein said status indicator comprises an LED.

15. The fluid treatment system of claim 1, further comprising a reflective material disposed in the conduit and configured to distribute UV radiation emitted by the UV emitter.

16. The fluid treatment system of claim 15, wherein said reflective material comprises at least one of aluminum or Teflon.

17. The fluid treatment system of claim 1, wherein said control unit is further configured to:
detect a failure of the active cleaning device; and
generate an alert indicating the active cleaning device has failed.

18. The fluid treatment system of claim 17, wherein to detect a failure of the active cleaning device, said control unit is configured to determine a measured parameter falls outside of a threshold range.

19. A method for operating a fluid treatment system to treat a fluid moving through a conduit, said method comprising:
energizing, using a control unit electrically coupled to an active cleaning device and to a power supply, the active cleaning device using the power supply to activate the active cleaning device to neutralize or remove at least a portion of an undesired matter from the fluid in response to a presence of fluid flow through the conduit; and
deenergizing, using the control unit, the active cleaning device to deactivate the active cleaning device in response to an absence of fluid flow through the conduit.

20. A fluid moving system comprising:
a conduit;
an active cleaning device configured to neutralize or remove at least a portion of an undesired matter from a fluid moving through said conduit;
a power supply configured to supply electric power to said active cleaning device; and
a control unit electrically coupled to said active cleaning device and to said power supply, said control unit configured to:
energize said active cleaning device using said power supply to activate said active cleaning device in response to a presence of fluid flow through said conduit; and
deenergizing said active cleaning device to deactivate said active cleaning device in response to an absence of fluid flow through said conduit.
